(19) United States
(12) Patent Application Publication
(10) Pub. No.: US 2026/0176218 A1
(43) Pub. Date: Jun. 25, 2026

(54) PROCESS FOR PRODUCING LIGHT OLEFINS

(71) Applicant: UOP LLC, Rosemont, IL (US)

(72) Inventors: Gregory A. Funk, Carol Stream, IL (US); Patrick C. Whitchurch, Sleepy Hollow, IL (US); David Lafyatis, Schaumburg, IL (US); John Anthony Petri, Wauconda, IL (US); Keith Allen Couch, Arlington Heights, IL (US); Alan K. Bedell, Durango, CO (US); Jonathan Roger Juan Jocelyn Schwob, Madinah (SA)

(21) Appl. No.: 19/418,574

(22) Filed: Dec. 12, 2025

Related U.S. Application Data
(60) Provisional application No. 63/734,100, filed on Dec. 14, 2024.

(51) Int. Cl. C07C 4/06; C07C 7/00

(52) U.S. Cl. CPC C07C 4/06; C07C 7/00

(57) ABSTRACT

A process for producing light olefins is disclosed. The process comprises hydrocracking a hydrocarbon feed stream in a hydrocracking reactor over a hydrocracking catalyst in the presence of a hydrocracking hydrogen stream at hydrocracking conditions to produce a hydrocracked stream. A reforming charge stream is taken from the hydrocracked stream. The reforming charge stream is reformed over a reforming catalyst in a reforming reactor to convert naphthenes to aromatics and produce a reformed stream. The reformed stream is charged to an NEP reactor to convert the reformed stream over an NEP catalyst in the presence of NEP hydrogen to produce a light paraffinic stream. Ethylene and propylene can be produced from the light paraffinic stream.

### FIELD

The field is related to a process producing light olefins. Particularly, the field is related to producing light olefins and aromatics.

### BACKGROUND

Hydroprocessing can include processes which convert hydrocarbons in the presence of hydroprocessing catalyst and hydrogen to more valuable products. Hydrocracking is a hydroprocessing process in which hydrocarbons crack in the presence of hydrogen and hydrocracking catalyst to lower molecular weight hydrocarbons. Depending on the desired output, a hydrocracking unit may contain one or more fixed beds of the same or different catalyst. Hydrotreating is a process in which hydrogen is contacted with a hydrocarbon stream in the presence of hydrotreating catalyst which is primarily active for the removal of heteroatoms, such as sulfur, nitrogen and metals from the hydrocarbon feed stream. In hydrotreating, olefinic hydrocarbons with double and triple bonds may be saturated. Aromatics may also be saturated. Some hydrotreating processes are specifically designed to saturate aromatics.

Two-stage hydrocracking processes involve fractionation of a hydrocracked stream from a first stage hydrocracking reactor followed by hydrocracking of an unconverted oil (UCO) stream in a second stage hydrocracking reactor. Typically, a bottoms stream from the fractionation column in two-stage hydrocracking comprises a recycle oil (RO) stream and an UCO stream. The RO is recycled to the second stage hydrocracking reactor while the UCO is purged from the process to remove unconvertible heavy polynuclear aromatics (HPNA's) from the process. HPNA's are fused aromatic rings comprising at least eight rings. HPNA's in RO and UCO can cause significant adverse impact on hydrocracking operations such as fouling of the exchangers and coking on the catalyst. Several processes are available to manage HPNA rejection, such as steam stripping and adsorption.

Light olefin production is vital to the production of sufficient plastics to meet worldwide demand. Ethylene and propylene are important chemicals for use in the production of other useful materials, such as polyethylene and polypropylene. Polyethylene and polypropylene are two of the most common plastics found in use today and have a wide variety of uses. Uses for ethylene and propylene include the production of vinyl chloride, ethylene oxide, ethylbenzene, cumene, polyols and alcohol.

Dehydrogenation is a process in which light paraffins such as ethane and propane can be dehydrogenated to make ethylene and propylene respectively, typically in the presence of a catalyst. Dehydrogenation can be achieved in either the presence of an oxidant such as oxygen or in the absence of an oxidant. Non-oxidative dehydrogenation is an endothermic reaction which requires external heat to drive the reaction to completion.

Fluid catalytic cracking (FCC) is another endothermic process that can be tuned to produce substantial propylene. However, not every FCC unit is tuned to make substantial propylene. Also, high propylene FCC units do not recover much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to the cracking furnace to reduce the hydrocarbon partial pressure and enhance olefin yield and to reduce the formation and deposition of carbonaceous material in the cracking reactors. The process is therefore often referred to as steam cracking or pyrolysis.

Paraffins with a range of carbon numbers can be thermally cracked to produce olefins including ethane, propane, butanes, and naphtha. Ethane and naphtha feeds are typical due to higher light olefin yield than propane and butane feeds. Ethane feed is used in regions where light hydrocarbon gases are prevalent. In regions where gas is not abundant, naphtha feed is employed for steam cracking. Naphtha steam cracking has long set the price in the ethylene industry due to higher production cost versus ethane steam cracking. The world does not currently produce enough ethane to supply the growing demand for ethylene. Therefore, regions lacking ethane supply such as Asia and Europe rely mainly on naphtha steam cracking to supply ethylene. Naphtha steam cracking yields only about 30%-35% ethylene with the balance including both relatively high-value by-products comprising propylene, butadiene, and butene-1 and relatively low value by-products comprising pyoil, pygas, and fuel gas. Additional pressures on naphtha steam cracking including minimum production requirements and environmental concerns have led to the withholding of government approvals in certain regions such as China.

Xylene isomers are important intermediates in chemical syntheses, and specific xylene isomers are desired for different processes. Paraxylene is a feedstock for terephthalic acid, and terephthalic acid is used in the manufacture of synthetic fibers and resins. Metaxylene is used in the manufacture of certain plasticizers, azo dyes, and wood preservatives. Orthoxylene is a feedstock for phthalic anhydride production, and phthalic anhydride is used in the manufacture of certain plasticizers, dyes, and pharmaceutical products. It is desirable to develop methods and systems for producing selected xylene isomers.

An improved process for producing the light olefins and the aromatics is needed to address the aforesaid limitations.

### BRIEF SUMMARY

The present disclosure provides a process for producing light olefins such as ethylene and propylene. The process comprises hydrocracking a hydrocarbon feed stream in a hydrocracking reactor over a hydrocracking catalyst in the presence of a hydrocracking hydrogen stream at hydrocracking conditions to produce a hydrocracked stream. Selected hydrocracked streams may be recycled to further hydrocracking. A reforming charge stream is taken from the hydrocracked stream. The reforming charge stream is reformed over a reforming catalyst in a reforming reactor to convert naphthenes to aromatics and produce a reformed stream. The reformed stream is charged to an NEP reactor to convert the reformed stream over an NEP catalyst in the presence of NEP hydrogen to produce a light paraffinic stream. Ethylene and propylene can be produced from the light paraffinic stream. The disclosed process provides flexibility for producing selected aromatics while producing the light olefins.

### BRIEF DESCRIPTION OF THE DRAWINGS

FIG. 1 is a schematic drawing of an exemplary embodiment of the process for producing light olefins.

FIG. 2 is a schematic drawing of another exemplary embodiment of the process for producing light olefins.

FIG. 3 is a schematic drawing of another exemplary embodiment of the process for producing light olefins.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take a main product from the bottom.

As used herein, the term “rich” can mean an amount of at least generally 50%, and preferably 70%, more preferably 90% or above by mass of a compound or class of compounds in a stream.

As used herein, the term “a component-rich stream” or “a stream rich in a component” means that the rich stream coming out of a vessel has a greater concentration of the component than any other stream from the vessel.

As used herein, the term “a component-lean stream” or “a stream lean in a component” means that the lean stream coming out of a vessel has a smaller concentration of the component than any other stream from the vessel.

As used herein, the term “initial boiling point” (IBP) means the temperature at which the sample begins to boil using a simulated distillation method of ASTM D-7169, ASTM D-86 or D 1160, or TBP, as the case may be.

As used herein, the term “end point” (EP) means the temperature at which the sample has all boiled off using a simulated distillation method of ASTM D-7169, ASTM D-86 or D 1160, or TBP, as the case may be.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx−” refers to molecules with x and preferably x and less carbon atoms. The term “Cx+” refers to molecules with x and preferably x and more carbon atoms.

The term “unit” is to be understood to refer to one or more process steps comprising a chemical transformation. At the heart of a unit is one or more catalytic reactors or separation vessels necessary to accomplish the transformation. A unit may further comprise additional separation vessels including fractionation column(s) to separate product streams. A unit may further comprise pretreatment steps for the chemical transformation. Taken together, “unit” comprises one or more reactors or separation vessels and separation steps and pretreatment steps, whether or not shown in the diagram or explicitly discussed in the specification.

The terms “T10” and “T90” are used here to characterize the volatility of a petroleum fraction. T10 and T90 refer to the temperatures for recovery of 10% and 90%, respectively, in distillation of petroleum products corrected to atmospheric pressure using a laboratory standard method of ASTM D-7169, ASTM D-86 or D 1160, or TBP, as the case may be.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “carbon number” refers to the number of carbon atoms per hydrocarbon molecule.

As used herein, the term “passing” includes “feeding” and means that the material passes from a conduit or vessel to an object.

As used herein, the prefix “bio” as used herein, refers to an association with a renewable resource of biological origin, such resources generally being exclusive of fossil fuels.

As used herein, the term “net” with respect to products means products in the desired boiling range excluding unconverted materials such as unconverted oil.

### DETAILED DESCRIPTION

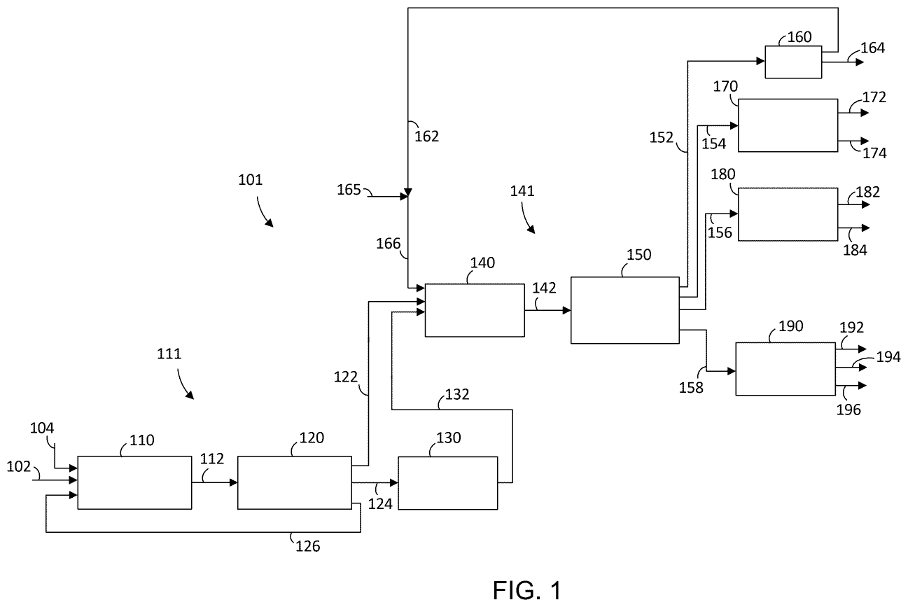

An embodiment of the process 101 for producing light olefins such as ethylene and propylene is shown in FIG. 1. The process 101 comprises a hydrocracking unit 111, a reforming unit 130, and a naphtha to ethane and propane (NEP) unit 141. In accordance with the present disclosure, the hydrocarbon feed stream in line 102 may be of petroleum origin or bio-based or a combination of both. In an aspect, the hydrocarbon feed stream in line 102 may comprise more than one feed stream. The hydrocarbon feed stream in line 102 may comprise one or more hydrocarbons or heteroatomic hydrocarbonaceous components such as C6 naphthenes, benzene and C7 through nominally C80 to C100. Such feeds may include heavy naphtha, kerosene, heavy diesel, distillates, gas oil and deasphalted oil from the distillation and/or extraction of crude oils. Other such feeds can include intermediates of the same aforementioned boiling ranges derived from refining processes such as delayed coking, flexicoking, slurry or ebullated bed, vacuum residue hydrocracking, fluidized catalytic cracking units and the like. Such feeds can also include plastic pyrolysis oils, Fischer-Tropsch liquids and waxes.

In accordance with the present disclosure, the hydrocarbon feed stream in line 102 may be hydroprocessed in several different non-limiting hydroprocessing configurations such as a once-through, single stage recycle and two-stage hydrocracking configurations. The number of stages employed can be somewhat, but not necessarily, economically dependent on the feed properties. For example, feed streams devoid or with minimal threshold organic nitrogen concentration could be processed in a once-through configuration or a single-stage recycle configuration. Feed stream with a higher than threshold nitrogen concentration would be more economically processed in a two-stage unit. In one example, a crude oil may be distilled into streams comprising heavy naphtha, kerosene, heavy diesel, atmospheric gas oil, vacuum gas oils and a vacuum residue. The vacuum residue may be further upgraded by extracting a deasphalted oil or the vacuum residue may be further converted into vacuum gas oil and lighter boiling products boiling higher than C6 naphthenes. One or more of the aforementioned distilled or extracted, streams upgraded from vacuum residue may be taken as the hydrocarbon feed stream in line 102. Aromatic-rich streams may also be taken as a feed stream or a component of the hydrocarbon feed stream in line 102. Plastics pyrolysis oil may also be taken as a feed stream or a component of the hydrocarbon feed stream in line 102.

In an embodiment, the hydrocarbon feed stream in line 102 may be processed in a single or a multistage hydrocracking unit 111. In an exemplary embodiment, the hydrocracking unit 111 may be a single-stage hydrocracking unit comprising a first hydrocracking reactor 110. A single-stage hydrocracking unit 111 may be selected for feed that is less aromatic and more paraffinic.

In an embodiment, the hydrocracking unit 111 may comprise a hydrocracking reactor 110 and a separation section 120. The hydrocarbon feed stream in line 102 and a hydrocracking hydrogen stream in line 104 is charged to the hydrocracking reactor 110. The hydrocracking feed stream in line 102 is hydrocracked in the hydrocracking reactor 110 over a hydrocracking catalyst in the presence of the hydrocracking hydrogen stream in line 104 at hydrocracking conditions to produce a hydrocracked stream. In an aspect, the hydrocracking unit 111 may comprise a hydrotreating reactor (not shown) for hydrotreating the hydrocracking feed stream.

Hydrotreating is a process wherein hydrogen is contacted with hydrocarbon in the presence of hydrotreating catalysts which are primarily active for the removal of heteroatoms, such as sulfur, nitrogen, chlorine, and metals from the hydrocarbon feed stream. In hydrotreating, olefinic hydrocarbons with double and triple bonds may be saturated. Aromatics may also be saturated. Some hydrotreating processes are specifically designed to saturate aromatics.

The hydrotreating reactor may comprise a guard bed of hydrotreating catalyst followed by one or more beds of higher activity hydrotreating catalyst. The guard bed filters particulates and reacts contaminants in the hydrocarbon feed stream such as organo-metallic components containing metals like nickel, vanadium, silicon and arsenic which load onto the catalyst and deactivate the catalyst. The guard bed may comprise material similar to the hydrotreating catalyst. Supplemental hydrogen may be added at an interstage location between catalyst beds in the hydrotreating reactor.

Suitable hydrotreating catalysts for use in the hydrotreating reactor may include any known conventional hydrotreating catalysts and include those which are comprised of at least one Group VIII metal, preferably iron, cobalt and nickel, more preferably cobalt and/or nickel and at least one Group VI metal, preferably molybdenum and tungsten, on a high surface area support material, preferably alumina. Other suitable hydrotreating catalysts include zeolitic catalysts. More than one type of hydrotreating catalyst may be used in the hydrotreating reactor. The Group VIII metal is typically present in an amount ranging from about 2 to about 20 wt-%, preferably from about 4 to about 12 wt-%. The Group VI metal will typically be present in an amount ranging from about 1 to about 25 wt-%, preferably from about 2 to about 25 wt-%.

Preferred reaction conditions in the hydrotreating reactor may include a temperature from about 290° C. (550° F.) to about 455° C. (850° F.), suitably 316° C. (600° F.) to about 427° C. (800° F.) and preferably 343° C. (650° F.) to about 399° C. (750° F.), a pressure from about 2.1 MPa (gauge) (300 psig), preferably 4.1 MPa (gauge) (600 psig) to about 20.6 MPa (gauge) (3000 psig), suitably 13.8 MPa (gauge) (2000 psig), preferably 12.4 MPa (gauge) (1800 psig), a liquid hourly space velocity of the fresh hydrocarbon feed stream from about 0.1 hr⁻¹, suitably 0.5 hr⁻¹, to about 10 hr⁻¹, preferably from about 1.5 to about 8.5 hr⁻¹, and a hydrogen rate of about 168 Nm3/m3 (1,000 scf/bbl), to about 1,011 Nm3/m3 oil (6,000 scf/bbl), preferably about 168 Nm3/m3 oil (1,000 scf/bbl) to about 674 Nm3/m3 oil (4,000 scf/bbl), with a hydrotreating catalyst or a combination of hydrotreating catalysts.

The hydrocarbon feed stream in line 102 may be hydrotreated over the hydrotreating catalyst to provide a hydrotreated hydrocarbon feed stream which can be taken as a hydrocracking feed stream. The hydrogen gas laden with ammonia and hydrogen sulfide may be removed from the hydrocracking feed stream in a separator, but the hydrocracking feed stream may be typically fed directly to the hydrocracking reactor 110 without separation.

Hydrocracking is a process in which hydrocarbons crack in the presence of hydrogen to lower molecular weight hydrocarbons. The hydrocracking reactor 110 may be a fixed bed reactor that comprises one or more vessels, single or multiple catalyst beds in each vessel, and various combinations of hydrotreating catalyst, hydroisomerization catalyst and/or hydrocracking catalyst in one or more vessels. The hydrocracking reactor 110 may be operated in a conventional continuous gas phase, a moving bed or a fluidized bed hydroprocessing reactor. More typically, the hydrocracking reactor 110 is operated in a mixed phase with gas and liquid phases passing over a stationary solid, fixed bed of catalyst or catalysts.

The hydrocracking reactor 110 may comprise a plurality of hydrocracking catalyst beds. If the hydrocracking unit 111 does not include a hydrotreating reactor, the first catalyst bed in the hydrocracking reactor 110 may include a hydrotreating catalyst for the purpose of saturating, demetallizing, desulfurizing, dechlorinating or denitrogenating the hydrocarbon feed stream in line 102 before it is hydrocracked over the hydrocracking catalyst in subsequent vessels or catalyst beds in the hydrocracking reactor 110. Otherwise, the first or an upstream bed in the hydrocracking reactor 110 may comprise a hydrocracking catalyst bed.

The hydrotreated hydrocracking feed stream is hydrocracked over the hydrocracking catalyst in the hydrocracking catalyst beds in the presence of the hydrocracking hydrogen stream in line 104 to provide a hydrocracked stream. Subsequent catalyst beds in the hydrocracking reactor 110 may comprise hydrocracking catalyst over which additional hydrocracking occurs to the hydrocracked stream. Supplemental hydrogen may be added to each of the hydrocracking catalyst beds at an interstage location between adjacent beds, so supplemental hydrogen is mixed with hydroprocessed stream exiting from the upstream catalyst bed before entering the downstream catalyst bed.

In the hydrocracking reactor 110, under the aforesaid prevalent hydrocracking conditions, a predominant proportion of the rings present in the hydrocarbon feed stream in line 102 may be opened to produce aliphatic hydrocarbons in a hydrocracked stream. In an exemplary embodiment, the rings present in the hydrocarbon feed stream in line 102 may comprise naphthene rings and aromatics rings.

The hydrocracking catalyst may utilize bases comprising amorphous silica-alumina or zeolites combined with one or more Group VIII or Group VIB metal hydrogenating components if mild hydrocracking is desired to produce a balance of middle distillate and gasoline. In another aspect, when light naphtha and LPG, which are aliphatic hydrocarbons or six carbon numbers and lower, are significantly preferred in the converted product over gasoline or distillate production, partial or complete hydrocracking conversion to aliphatic hydrocarbons of six carbon numbers or less may be performed in the hydrocracking reactor 110 with a catalyst which comprises, in general, any crystalline zeolite cracking base upon which is deposited a Group VIII metal hydrogenating component. Additional hydrogenating components may be selected from Group VIB for incorporation with the zeolite base. In one embodiment, when the hydrocracking reactor 110 is operated in a single-stage reaction configuration, complete hydrocracking conversion to aliphatic hydrocarbons of six carbon numbers or less may be performed.

The zeolites of hydrocracking bases are sometimes referred to in the art as molecular sieves and are usually composed of silica, alumina and one or more exchangeable cations such as sodium, magnesium, calcium, rare earth metals, etc. They are further characterized by crystal pores of relatively uniform diameter between about 4 and about 14 Angstroms. It is preferred to employ zeolites having a relatively high silica/alumina mole ratio between about 3 and about 12. Suitable zeolites found in nature include, for example, mordenite, stilbite, heulandite, ferrierite, dachiardite, chabazite, erionite and faujasite. Suitable synthetic zeolites include, for example, the B, X, Y and L crystal types, e.g., synthetic faujasite and mordenite. The preferred zeolites are those having crystal pore diameters between about 8 and 12 angstroms, wherein the silica/alumina mole ratio is about 4 to 6. One example of a zeolite falling in the preferred group is synthetic Y molecular sieve.

The natural occurring zeolites are normally found in a sodium form, an alkaline earth metal form, or mixed forms. The synthetic zeolites are nearly always prepared first in the sodium form. In any case, for use as a cracking base it is preferred that most or all of the original zeolitic monovalent metals be ion-exchanged with a polyvalent metal and/or with an ammonium salt followed by heating to decompose the ammonium ions associated with the zeolite, leaving in their place hydrogen ions and/or exchange sites which have actually been decationized by further removal of water. Hydrogen or “decationized” Y zeolites of this nature are more particularly described in U.S. Pat. No. 3,100,006.

Mixed polyvalent metal-hydrogen zeolites may be prepared by ion-exchanging first with an ammonium salt, then partially back exchanging with a polyvalent metal salt and then calcining. In some cases, as in the case of synthetic mordenite, the hydrogen forms can be prepared by direct acid treatment of the alkali metal zeolites. In one aspect, the preferred cracking bases are those which are at least about 10 wt-%, and preferably at least about 20 wt-%, metal-cation-deficient, based on the initial ion-exchange capacity. In another aspect, a desirable and stable class of zeolites is one wherein at least about 20 wt-% of the ion exchange capacity is satisfied by hydrogen ions.

The active metals employed in the preferred first hydrocracking catalysts of the present invention as hydrogenation components are those of Group VIII, i.e., iron, cobalt, nickel, ruthenium, rhodium, palladium, osmium, iridium and platinum. In another embodiment, Group VIII metals such as nickel or cobalt may be used to promote the aromatic saturation and intermediates re-hydrogenation activity of Group VIB metals. Such Group VIB hydrogenation metals may comprise molybdenum and tungsten. The amount of hydrogenating metal in the catalyst can vary within wide ranges. Broadly speaking, any amount between about 0.05 wt-% and about 30 wt-% may be used. In the case of noble metals, it is normally preferred to use about 0.05 to about 2 wt-% noble metal.

The method for incorporating the hydrogenation metal is to contact the base material with an aqueous solution of a suitable compound of the desired metal. Following addition of the selected hydrogenation metal or metals, the resulting catalyst powder is then filtered, dried, pelleted with added lubricants, binders or the like if desired, and calcined in air at temperatures of, e.g., about 371° C. (700° F.) to about 648° C. (1200° F.) in order to activate the catalyst and decompose ammonium ions. Alternatively, the base component may first be pelleted, followed by the addition of the hydrogenation metal and activation by calcining. In yet another embodiment the base component may first be pelleted, followed by the addition of the hydrogenation metal and dried.

The foregoing catalysts may be employed in undiluted form, or the powdered catalyst may be mixed and copelleted with other relatively less active catalysts, diluents or binders such as alumina, silica gel, silica-alumina cogels, activated clays and the like in proportions ranging between about 5 and about 90 wt-%. These diluents may be employed as such or they may contain a minor proportion of an added hydrogenating metal such as a Group VIB and/or Group VIII metal. Additional metal promoted hydrocracking catalysts may also be utilized in the process of the present invention which comprises, for example, aluminophosphate molecular sieves, crystalline chromosilicates and other crystalline silicates. Crystalline chromosilicates are more fully described in U.S. Pat. No. 4,363,718.

In an embodiment, the hydrocracking conditions in the hydrocracking reactor 110 may include a temperature from about 290° C. (550° F.) to about 468° C. (875° F.), preferably 343° C. (650° F.) to about 445° C. (833° F.), a pressure from about 4.8 MPa (gauge) (700 psig) to about 20.7 MPa (gauge) (3000 psig), a liquid hourly space velocity (LHSV) of about 0.3 to about 1.5 hr⁻¹, suitably no more than about 1.0 hr⁻¹ and preferably about 0.4 to about 0.7 hr⁻¹ and a hydrogen rate of about 421 Nm3/m3 (2,500 scf/bbl) to about 2,527 Nm3/m3 oil (15,000 scf/bbl). Hydrogen partial pressure may be 1 MPa (abs) (1500 psia) to about 1.7 MPa (abs) (2500 psia).

A hydrocracked stream in line 112 is discharged from the hydrocracking reactor 110. The hydrocracked stream in line 112 is passed to the separation section 120. The separation section 120 may comprise one or more of a hot separator, a cold separator, flash drums, and fractionation column.

The hot separator separates the hydrocracked stream to provide a hot gaseous stream and a hot liquid stream. The hot separator may be operated at about 177° C. (350° F.) to about 371° C. (700° F.) and preferably operates at about 232° C. (450° F.) to about 315° C. (600° F.). The hot separator may be operated at a slightly lower pressure than the hydrocracking reactor 110 accounting for pressure drop through intervening equipment. The hot separator may be operated at pressures between about 3.4 MPa (gauge) (493 psig) and about 20.4 MPa (gauge) (2959 psig).

The hot gaseous stream may be separated in the cold separator to provide a cold hydrogen-rich gas stream and a cold liquid stream. The cold separator serves to separate hydrogen rich gas from hydrocarbon liquid in the hydrocracked stream for recycle to the hydrocracking unit 111. The cold separator may be operated at about 100° F. (38° C.) to about 150° F. (66° C.), suitably about 115° F. (46° C.) to about 145° F. (63° C.), and below the pressure of the hydrocracking reactor 110 and the hot separator accounting for pressure drop through intervening equipment to keep hydrogen and light gases in the overhead and normally liquid hydrocarbons in the bottoms. The cold separator may be operated at pressures between about 3 MPa (gauge) (435 psig) and about 20 MPa (gauge) (2,901 psig). The cold separator may also have a boot for collecting an aqueous phase.

The cold gaseous stream is rich in hydrogen. The cold gaseous stream may be passed through a trayed or packed recycle scrubbing column where it is scrubbed by means of a scrubbing extraction liquid such as an aqueous solution to remove acid gases including hydrogen sulfide and carbon dioxide by extracting them into the aqueous solution. Preferred aqueous solutions include lean amines such as alkanolamines DEA, MEA, and MDEA. Other amines can be used in place of or in addition to the preferred amines. The lean amine contacts the cold gaseous stream and absorbs acid gas contaminants such as hydrogen sulfide and carbon dioxide. The resultant “sweetened” cold gaseous stream is taken out from the recycle scrubber column, and a rich amine is taken out from the bottoms of the recycle scrubber column. The spent scrubbing liquid may be regenerated and recycled back to the recycle scrubbing column. The scrubbed hydrogen-rich stream emerges from the scrubber, may be supplemented with make-up hydrogen stream, and recycled to the hydrocracking unit 111. The scrubbed hydrogen-rich stream may be compressed before recycling to the hydrocracking unit 111. The recycle scrubbing column may be operated with a gas inlet temperature between about 38° C. (100° F.) and about 80° C. (175° F.) and an overhead pressure of about 3 MPa (gauge) (435 psig) to about 20 MPa (gauge) (2900 psig).

The hot liquid stream may be directly stripped. In an aspect, the hot liquid stream may be let down in pressure and flashed in a hot flash drum to provide a flash hot gaseous stream of light ends and a flash hot liquid stream. In an aspect, light gases such as hydrogen sulfide may be stripped from the flash hot liquid stream. The hot flash drum may be operated at the same temperature as the hot separator but at a lower pressure of between about 1.4 MPa (gauge) (200 psig) and about 6.9 MPa (gauge) (1000 psig), suitably no more than about 3.8 MPa (gauge) (550 psig). The flash hot liquid stream may be further fractionated in a fractionation section.

The cold liquid stream may be directly stripped. In a further aspect, the cold liquid stream may be let down in pressure and flashed in a cold flash drum. The flash hot gaseous stream may be cooled and also separated in the cold flash drum to provide a flash cold gaseous stream and a flash cold liquid stream. The cold flash drum may be operated at the same temperature as the cold separator 128 but typically at a lower pressure of between about 1.4 MPa (gauge) (200 psig) and about 6.9 MPa (gauge) (1000 psig) and preferably between about 3.0 MPa (gauge) (435 psig) and about 3.8 MPa (gauge) (550 psig).

The fractionation section may include the stripping column and a naphtha splitter column. The flash cold liquid stream may be stripped of gases in the stripping column with a stripping media which is an inert gas such as steam to provide a stripper gaseous stream of naphtha, hydrogen, hydrogen sulfide, steam and other gases. A net stripper overhead stream may be taken for recovery of C1-C3 hydrocarbons. The stripping column may be operated with a bottoms temperature between about 149° C. (300° F.) and about 360° C. (680° F.) or about 160° C. (320° F.) to about 288° C. (550° F.), and an overhead pressure of about 0.35 MPa (gauge) (50 psig), preferably no less than about 0.50 MPa (gauge) (72 psig), to no more than about 2.0 MPa (gauge) (290 psig).

The flash hot liquid stream may be fed to the stripping column near a bottom half of the column. The flash hot liquid stream may be stripped in the stripping column of gases with a stripping media which is an inert gas such as steam. A liquid stripped stream is taken from the stripper bottoms. A reboiling stream may be taken from the liquid stripped stream and a net liquid stripped stream may be passed to the naphtha splitter column.

The naphtha splitter column may comprise more than one fractionation column for separating stripped hydrocracked streams into product streams. The naphtha splitter column may fractionate hydrocracked stream in the liquid stripped stream to provide the product streams. The product streams from the naphtha splitter column may include a splitter net overhead stream comprising liquefied petroleum gas and light naphtha and a heavy product stream. The heavy product stream may comprise heavy naphtha and unconverted oil stream comprising C6 naphthenes, and unconverted material with boiling points higher than benzene. All or a portion of the bottoms heavy product stream may be recycled to the hydrocracking unit 111 for further hydrocracking. A side product stream may also be taken from the naphtha splitter column. The naphtha splitter column may be operated with a bottom temperature between about 260° C. (500° F.), and about 385° C. (725° F.), preferably at no more than about 350° C. (650° F.), and at an overhead pressure between about 7 kPa (gauge) (1 psig) and about 69 kPa (gauge) (10 psig).

Typically, the heavy product which may be heavy naphtha and C6 aromatics are recycled back to the hydrocracking reactor. The present process provides selecting and adjusting cut point of one or more fractions from the fractionation section to allow some specific aromatics to be processed downstream of the hydrocracking unit 111 instead of recycling back to the hydrocracking unit. Further, the recycle stream to the hydrocracking reactor may be selected to provide fractions comprising selected hydrocarbons. These fractions are taken from the fractionation section and processed further downstream for producing the light olefins. The cut point of the fractionation section may be adjusted by adjusting operating conditions of the stripping column and the naphtha splitter column of the fractionation section. Further, the operating conditions of the hydrocracking reactor 110 may be optimized to affect the conversion of the feed into the selected hydrocarbons which can be taken as products streams or the recycle stream. The process provides flexibility for taking different fractions from the hydrocracking unit 111 which can be recycled to the hydrocracking unit 111, to either maximize or minimize specific aromatics depending on the customer's preference and balance the customer's need for producing the light olefins.

In accordance with the present disclosure, the cut point of the fractionation section may be adjusted to provide a hydrocracked paraffinic stream in line 122, a reforming charge stream in line 124, and a recycle stream in line 126. The reforming charge stream in line 124 may comprise a product stream from the hydrocracking unit 111. In an embodiment, the reforming charge stream in line 124 may comprise a light ends product stream from the hydrocracking unit 111. In an aspect, the hydrocracked paraffinic stream in line 122 may comprise paraffins with no more than 6 carbon atoms.

In an aspect, the hydrocracked paraffinic stream in line 122 may be an overhead stream taken from the fractionation section, the reforming charge stream in line 124 may be a side stream taken from the fractionation section, and the recycle stream in line 126 may be a bottom stream taken from the fractionation section.

In an embodiment, the fractionation section may comprise two fractionation columns to produce the hydrocracked paraffinic stream in line 122, the reforming charge stream in line 124, and the recycle stream in line 126.

In the embodiment as shown in FIG. 1, the cut point of the fractionation section may be adjusted to provide the reforming charge stream comprising specific hydrocarbons in line 124 which is fed to the reforming unit 130. The recycle stream in line 126 may comprise selected hydrocarbons which are recycled to the hydrocracking reactor 110. In an aspect, carbon range of the reforming charge stream in line 124 may depend on the cut point of the recycle stream in line 126. In an exemplary embodiment as shown in FIG. 1, the reforming charge stream in line 124 may comprise naphthenes and aromatics with six carbon atoms, and the recycle stream in line 126 may comprise C7+ hydrocarbons. Hence, the cut between the overhead stream and the side stream can be taken between C6− aliphatics in the overhead stream and C6 cyclics and aromatics in the side stream. Hydrocarbons of seven carbon atoms and greater can be taken in line 126 and recycled to the hydrocracking reactor 110.

In an embodiment, the reforming unit 130 comprises a reforming reactor. The reforming charge stream in line 124 which is a reforming charge stream, is charged to the reforming reactor. The reforming reactor may comprise one or more charge heaters and reactor vessels for reforming the reforming charge stream in line 124. In the reforming reactor the reforming charge stream is contacted with a reforming catalyst to convert C6 naphthenes to aromatics and produce a reformed stream. In an aspect, the reformed stream is an aromatics rich stream.

In the reforming reactor, the reforming charge stream in line 124 may contact the catalyst in individual reactors in either upflow, downflow, or radial flow fashion, with the radial flow mode being preferred. The catalyst is contained in a fixed-bed system or a moving-bed system with associated catalyst regeneration such as a semi-regeneration. The reforming reactor may be operated at a temperature of from about 400° C. to about 6000° C., or from about 450° C. to about 560° C. The reforming reactor may be operated at a pressure of about 413 kPa (gauge) (60 psig) to about 2758 kPa (gauge) (400 psig) or a lower pressure. The reforming reactor converts the paraffinic and naphthenic materials to aromatics to produce a reformed stream comprising a higher concentration of C₆-C₁₁ aromatics than the reforming charge stream in line 124.

Suitable catalysts for use in the reforming reactor may include a dual-function catalyst having a multi-metallic, combination of two or more metal components in specified concentrations on the finished catalyst, and its use in hydrocarbon conversion with increased aromatics production. Catalysts having both a hydrogenation-dehydrogenation function and a cracking function should maximize the former and minimize the latter. The cracking function generally relates to an acid-action material of the porous, adsorptive, refractory-oxide type which is typically utilized as the support or carrier for a heavy-metal component, such as the Group VIII (IUPAC 8-10) metals, which primarily contribute the hydrogenation-dehydrogenation function. Other metals in combined or elemental form can influence one or both of the cracking and hydrogenation-dehydrogenation functions.

Catalytic reforming involves a number of competing processes or reaction sequences. These include dehydrogenation of cyclohexanes to aromatics, dehydroisomerization of alkylcyclopentanes to aromatics, dehydrocyclization of an acyclic hydrocarbon to aromatics, dealkylation of alkylbenzenes and isomerization of paraffins. Hydrocracking reactions which produce light paraffin gases have a deleterious effect on the yield of products boiling in the gasoline or jet fuel range. Process improvements in catalytic reforming thus are targeted toward enhancing those reactions effecting a higher yield of the liquid products containing more 5 or more carbon atoms and minimizing those reactions affecting cracked products containing 4 or fewer carbon atoms.

Generally, it is desirable to have flexibility with reforming catalyst functionality. In one exemplary reforming process, increasing the yield of one or more C₅⁺ hydrocarbons, hydrogen, and aromatic yields is desired. Optionally, the acidity of the catalyst can be altered by adding a metal and/or other elements to the catalyst. Generally, modification of the acid function results in reduced cracking of the alkanes to C₃ and C₄ light ends allowing increased selectivity to the formation of aromatics. Modification of the metal function may also occur resulting in the reduction of alkane cracking to methane and ethane. There can also be a reduction in the dealkylation reactions of aromatics leaving heavier and more valuable C₈⁺ aromatics.

Beside the yields, the activity of a catalyst may enable obtaining a commercially useful conversion level without employing additional quantities of catalyst or using excessively high temperatures, which can lead to undesired higher costs. Higher catalyst activity can also be utilized to process greater quantities of feed or to increase conversion, and therefore increase the production of valuable products.

Catalytic materials used for reforming paraffinic feeds more selectively towards aromatics can be achieved by tuning the material acidity. In one embodiment, the catalytic material comprises a refractory aluminum oxide support, a metal from the platinum group, such as Pt, and a halogen element.

In an embodiment, the catalysts for use in the reforming reactor may comprise a zeolite-based catalyst with greater than 0 up to about 2 wt % of a noble metal, or a chlorinated alumina-based catalyst with greater than 0 up to about 1 wt % of a noble metal and one or more metals from tin, germanium, gallium, indium, and rhenium, or combinations thereof.

A reformed stream comprising an increased concentration of aromatics leaves the reforming reactor in line 132. In an aspect, the reformed stream in line 132 may comprise greater than about 40 wt-%, or greater than about 50 wt-%, suitably greater than 70 wt-% or preferably greater than about 90 wt-% aromatic hydrocarbons.

The reforming charge stream in line 124 comprises naphthenes and aromatics with six carbon atoms which may be suitable as benzene precursors to the reforming reaction. In the embodiment shown in FIG. 1, the reforming reactor may be operated at conditions including the temperature, pressure and suitable catalyst to maximize the production of benzene. In an exemplary embodiment, the reformed stream in line 132 may comprise about 20 wt % to about 95 wt % benzene.

The reformed stream in line 132 may comprise hydrogen, light ends such as C5-hydrocarbons, and unconverted paraffins along with the aromatics. In an aspect, the reformed stream in line 132 may comprise some lower hydrocarbons such as C6− paraffins along with the aromatics. The lower carbon number paraffins can be a feed source for a catalytic naphtha cracking process, such as the naphtha to ethane and propane (NEP) process. The NEP process may convert the light paraffins into ethane and propane. The reformed stream in line 132 and the hydrocracked paraffinic stream in line 122 may be charged to the NEP unit 141. As shown in FIG. 1, the hydrocracked paraffinic stream in line 122 may bypass the reforming unit 130 and fed directly to the NEP unit 141. In an alternate embodiment, all or a portion of the hydrocracked paraffinic stream in line 122 may be fed to the reforming unit 130.

The NEP unit 141 may comprise a NEP reactor 140 and a NEP separation section 150. The reformed stream in line 132 and the hydrocracked paraffinic stream in the overhead line 122 may be charged to the NEP reactor 140. In an embodiment, the hydrocracked paraffinic stream in line 122 may be passed to the reforming unit 130 and then to the NEP unit 141 because paraffinic molecules of less than six carbons are relatively inert in a reforming reactor. An NEP hydrogen stream in line 166 may be charged to the NEP reactor 140. In an embodiment, the reformed stream in line 132, the hydrocracked paraffinic stream in line 122, and the NEP hydrogen stream in line 166 may be combined to provide an NEP charge stream which is passed to the NEP reactor 140. In the NEP reactor 140, the reformed stream in line 132 and the hydrocracked paraffinic stream in line 122 may be contacted with a NEP catalyst to produce a light paraffinic stream.

Operating conditions in the NEP reactor 140 may include a reaction temperature of about 300° C. to about 600° C., suitably between about 325° C. and about 550° C., and preferably between about 350° C. and about 525° C. A total pressure in the NEP reactor 140 should be about 0.1 to about 3 MPa (abs), preferably greater than 1 MPa (abs).

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙAlₙSi₉₆-nO₁₉₂·16H₂O (0<n<10). In various embodiments, the ZSM-5 zeolite may comprise a silica-to-alumina molar ratio of 20 to 1000, 20 to 800, 20 to 600, 20 to 400, 20 to 200 or 20 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicon, zinc, titanium, zirconium and mixtures of thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce ⅛-inch to about 1/32-inch diameter calcined supports. Alternatively, the zeolite may be mixed with a silicon oxide containing binder and extruded to 1/32 to ¼ inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. In a further example, the metal may comprise platinum, palladium, iridium, rhenium, ruthenium and mixtures thereof. The metal may be a noble metal. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each of the transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least 60 wt %, suitably at least 70 wt %, preferably at least 80 wt % and most preferably at least 90 wt % of the metal is on the binder. The zeolite and/or the entire NEP catalyst is steam oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts must be reduced to activate them for catalyzing the NEP reaction. For example, the catalyst may be reduced in flowing hydrogen at about 500 to about 550° C. for about 2 to about 4 hours before contacting a feed.

After paraffin conversion, a light paraffinic stream is discharged from the NEP reactor 140 in line 142. The light paraffinic stream may comprise at least about 40 wt % ethane or at least about 40 wt % propane or at least about 70 wt % and preferably at least about 80 wt % ethane and propane.

In an embodiment shown in FIG. 1, the NEP reactor 140, under the aforesaid reaction conditions, may also produce some toluene and xylene which is discharged in the light paraffinic stream in line 142. In an aspect, the light paraffinic stream in line 142 as shown in FIG. 1 may comprise hydrogen, methane, benzene, toluene and xylenes along with the ethane and propane. The light paraffinic stream in line 142 may be passed to the NEP separation section 150 to separate the components in one or more streams. The light paraffinic stream in line 142 may be cooled and fed to the NEP separation section 150.

The NEP separation section may be a fractionation column or a series of fractionation columns and other separation units. The NEP separation section may comprise a demethanizer column that separates the light paraffinic stream into a gas stream in an overhead line 152 and a C2+ paraffin stream in a bottoms line. The gas stream in line 152 may be sent to a hydrogen purification unit such as a PSA unit 160 to recover hydrogen for recycle to the NEP reactor. A hydrogen rich stream in line 162 is discharged from the PSA unit 160. Remaining methane from the hydrogen purification unit may be discharged in line 164 for fuel gas. The hydrogen rich stream in line 162 may be combined with a make-up hydrogen stream in line 165 to provide the NEP hydrogen stream in line 166.

The C2+ paraffin stream may be fed to a deethanizer column to produce an ethane stream in a deethanizer overhead line 154 and a C3+ paraffin stream in a deethanized bottoms stream. The C3+ paraffin stream may then be fed to a depropanizer column to produce a propane stream in a depropanizer overhead line 156 and a heavy stream which may comprise C4+ hydrocarbons. The heavy stream is discharged in line 158 from the NEP separation section 150. In the embodiment as shown in FIG. 1, the heavy stream in line 158 is an aromatic stream and may comprise benzene, toluene and xylenes. The NEP separation section 150 may take other forms.

The ethane stream is discharged in line 154 from the NEP unit 141. The ethane stream in line 154 may be charged to an ethylene producing unit 170 in which ethane in the ethane stream is converted into ethylene. In an embodiment, the ethylene producing unit 170 is a steam cracking unit. The ethane stream in line 154 may be cracked under steam in a furnace to produce a cracked stream including an ethylene stream. The ethane stream may be charged to the ethane steam cracking unit in the gas phase. The ethane steam cracking unit may preferably be operated at a temperature of about 750° C. (1382° F.) to about 950° C. (1742° F.). An ethylene stream is discharged in line 174 from the steam cracking unit 170. A hydrogen containing stream may be discharged in line 172 from the steam cracking unit 170.

The propane stream is discharged in line 156 from the NEP unit 141. The propane stream in line 156 may be charged to a propylene producing unit 180 in which propane in the propane stream is converted into propylene. The C3− hydrocarbon stream in line 156 may be charged to the propylene producing unit 180 to convert C3 into propylene. The propylene producing unit 180 may be a propane dehydrogenation (PDH) unit. PDH catalyst is used in a dehydrogenation reaction process to catalyze the dehydrogenation of propane. The conditions in the dehydrogenation reactor may include a temperature of about 500° C. to about 800° C., a pressure of about 40 to about 310 kPa (abs) and a catalyst to oil ratio of about 5 to about 100.

The dehydrogenation reaction may be conducted in a fluidized manner such that gas, which may comprise the reactant paraffins with or without a fluidizing inert gas, is distributed to the reactor in a way that lifts the dehydrogenation catalyst in the reactor vessel while catalyzing the dehydrogenation of paraffins. During the catalytic dehydrogenation reaction, coke is deposited on the dehydrogenation catalyst leading to reduction of the activity of the catalyst. The dehydrogenation catalyst must then be regenerated in a regenerator. The regenerator may combust coke from the dehydrogenation catalyst and fuel gas to ensure sufficient enthalpy in the dehydrogenation reactor to promote the endothermic reaction.

The dehydrogenation catalyst selected should minimize cracking reactions and favor dehydrogenation reactions. Suitable catalysts for use herein include an active metal which may be dispersed in a porous inorganic carrier material such as silica, alumina, silica alumina, zirconia, or clay. An exemplary embodiment of a catalyst includes alumina or silica-alumina containing gallium, a noble metal, and an alkali or alkaline earth metal.

The catalyst support comprises a carrier material, a binder and an optional filler material to provide physical strength and integrity. The carrier material may include alumina or silica-alumina. Silica sol or alumina sol may be used as the binder. The alumina or silica-alumina generally contains alumina of gamma, theta and/or delta phases. The catalyst support particles may have a nominal diameter of about 400 to about 5000 micrometers with the average diameter of about 600 to about 3500 micrometers. Preferably, the surface area of the catalyst support is about 85 to about 140 m²/g.

The fluidized dehydrogenation catalyst may comprise a dehydrogenation metal on a support. The dehydrogenation metal may be a one or a combination of transition metals. A noble metal may be a preferred dehydrogenation metal such as platinum or palladium. Gallium is an effective metal for paraffin dehydrogenation. Metals may be deposited on the catalyst support by impregnation or other suitable methods or included in the carrier material or binder during catalyst preparation.

The acid function of the catalyst should be minimized to prevent cracking and favor dehydrogenation. Alkali metals and alkaline earth metals may also be included in the catalyst to attenuate the acidity of the catalyst. Rare earth metals may be included in the catalyst to control the activity of the catalyst. Concentrations of 0.001% to 10 wt % metals may be incorporated into the dehydrogenation catalyst. In the case of the noble metals, it is preferred to use about 10 parts per million (ppm) by weight to about 600 ppm by weight noble metal. More preferably it is preferred to use about 10 to about 100 ppm by weight noble metal. The preferred noble metal is platinum. Gallium should be present in the range of 0.3 wt % to about 3 wt %, preferably about 0.5 wt % to about 2 wt %. Alkali and alkaline earth metals may be present in the range of about 0.05 wt % to about 1 wt %.

Regenerated catalyst may be contacted with the propane in line 156 and perhaps with a fluidizing gas to lift the propane stream and dehydrogenation catalyst up a riser while dehydrogenation occurs. Above the riser spent dehydrogenation catalyst and propylene product may be separated by a centripetal separation device. Propylene product gas may be quenched with a cooling fluid to prevent over reaction to undesired by-products. Separation of the propylene product from the PDH effluent stream may include quench contacting and fractionation to produce a propylene product stream. Unreacted propane may be recycled to the dehydrogenation reactor and lighter gases may be recycled to the regenerator as fuel gas to be combusted to provide enthalpy for the reaction.

The propylene producing unit may also employ a catalytic moving bed reactor. The reactor section may comprise several radial flow reactors in parallel or series heated by charge and interstage heaters. The propane stream perhaps with added hydrogen flows in each dehydrogenation reactor from a screened center pipe through an annular dehydrogenation catalyst bed to an outer effluent annulus. Flow may be in the reverse fashion. The dehydrogenation catalyst may comprise a noble metal or mixtures thereof, a modifier selected from the group consisting of alkali metals or alkaline-earth metals and mixtures thereof, a component selected from the group consisting of tin, germanium, lead, indium, gallium, thallium, and mixtures thereof, and a porous support forming a catalyst particle. The catalyst support may comprise oil dropped alumina spheres.

Dehydrogenation conditions may include a temperature of from about 400 to about 900° C., a pressure of from about 0.01 to 10 atmospheres absolute, and a liquid hourly space velocity (LHSV) of from about 0.1 to 100 hr−1. In an embodiment, the dehydrogenation conditions may include a reactor inlet temperature of from about 580 to about 640° C. and a reactor outlet pressure of about atmospheric pressure. The pressure in the dehydrogenation reactor is maintained as low as practicable, consistent with equipment limitations, to maximize chemical equilibrium advantages. Spent dehydrogenation catalyst in the annular catalyst bed may be withdrawn from the bottom of the bed, forwarded to a regenerator to combust coke from the catalyst with air at about 450 to about 600° C. Noble metal on the catalyst may be redispersed by an oxyhalogenation process, dried and returned to the top of the dehydrogenation catalyst bed as regenerated dehydrogenation catalyst.

Dehydrogenation effluent may be cooled, compressed, dried and hydrogen is cryogenically separated from the hydrocarbons with a net gas purity of 85 to 93 mol % hydrogen. Hydrocarbon liquid is selectively hydrogenated to convert diolefins and acetylenes and the hydrocarbon liquid is fractionated in a deethanizer column to remove ethane and propylene is split from propane in a propane-propylene splitter column to provide polymer-grade propylene. Propane may be recycled as feed to the propylene producing unit 170. A propylene stream is discharged in line 184 from the PDH unit 180. A hydrogen containing stream may be discharged in line 182 from the PDH unit 180.

In the embodiment as shown in FIG. 1, the heavy stream in line 158 may be charged to the aromatic complex 190 to sperate aromatics, particularly benzene toluene and xylenes. The aromatics complex 190 may comprise some or all of an aromatic extraction unit, a clay treater, a BTX fractionation zone, a transalkylation unit, a xylene fractionation zone, a xylene extraction unit, a heavy aromatics column, and an isomerization unit.

The heavy stream in line 158 may be passed to the clay treater for the removal of any alkylates and olefins that may be present in the heavy stream. A treated heavy stream may be passed to the xylene fractionation zone. The xylene fractionation zone may comprise a xylene column operated at conditions suitable for producing an overhead xylene containing stream. The xylene containing stream may comprise more than about 98 wt-% mixed xylenes. The xylene fractionation zone also produces a heavy bottom stream comprising C9+ hydrocarbons. The heavy bottom stream may be fed to the heavy aromatics column to provide a heavy aromatics overhead stream comprising C9 and C10 aromatics that is fed to the transalkylation unit. The heavy aromatics column also produces a heavy aromatics bottom stream comprising C11+ hydrocarbons. The heavy aromatics column may be operated at a pressure of about 5 kPa (0.7 psia) to about 1,800 kPa (260 psia) and a temperature of about 100° C. (212° F.) to about 360° C. (680° F.).

The xylene containing stream may be introduced into a xylene extraction unit that extracts a selected xylene isomer from non-selected xylene isomers that comprises a xylene raffinate stream. The xylene extraction unit may be based on an adsorption process or a crystallization process or any combination of both. In an aspect, the xylene extraction unit may include a selective adsorbent that preferentially sorbs the selected xylene isomer relative to the other xylene isomers. The xylene raffinate stream may be fed to an isomerization unit to isomerize the non-selected xylene isomers to produce more of the selected xylene isomer. In an embodiment, the selected xylene isomer is paraxylene. The isomerized effluent from the isomerization unit may be recycled to the xylene fractionation zone. The selected xylene isomer stream exits the xylene extraction unit and discharged from the aromatic complex 190 in selected xylene line 196.

A transalkylation effluent stream including benzene and toluene exits the transalkylation unit and may thereafter be passed to the BTX fractionation zone. The BTX fractionation zone may comprise two columns with the benzene stream and the concentrated toluene stream in serial overhead lines and the xylene stream in the second bottoms line. In an embodiment, the BTX fractionation zone may comprise a split shell distillation column for the separation of the benzene stream in a benzene line from the concentrated toluene stream in the concentrated toluene line and the xylene stream in xylene line. The benzene stream, having a lower boiling point than toluene, may be removed from the distillation column as an overhead product. The benzene stream may be discharged in line 192 from the aromatic complex 190. The toluene, having a higher boiling point than benzene, may be removed from distillation column as a sidedraw product. The toluene stream may be discharged in line 194 from the aromatic complex 190. Also, a net bottoms liquid stream including heavier aromatic hydrocarbons such as various xylene isomers, is removed from the BTX fractionation zone and thereafter fed to the xylene fractionation zone.

The process 101 may integrate multiple units and provides selectively producing one or more aromatics stream while producing the light olefins form the process. The process simplifies the separation section of the hydrocracking unit 111 and avoids the need for a dedicated separation section for the reforming unit 130. The process further improves the overall aromatic yield from the reforming unit 130. Furthermore, the process may improve the methyl-phenyl ratio to aromatics complex 190 which enables application of less costly liquid-phase isomerization unit and enables lower co-production of benzene as required. The process may significantly reduce the capital expenditure of the overall unit when aromatics production is required while producing the light olefins.

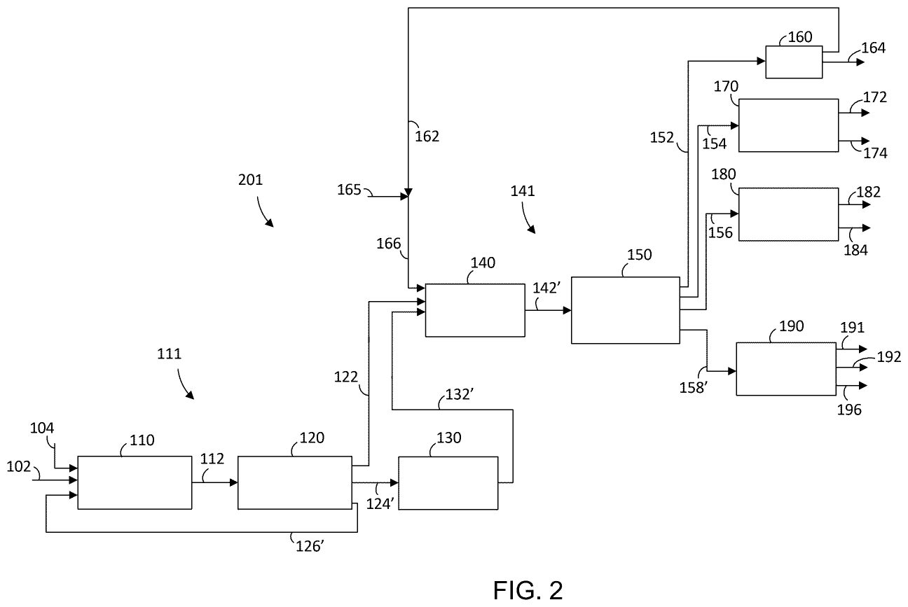

Another exemplary embodiment of the process for producing light olefins 201 is shown in FIG. 2. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is essentially the same as in FIG. 1 with the following exceptions.

In the embodiment shown in FIG. 2, the cut point of the reforming charge stream in line 124′ and the recycle stream in line 126′ are selected from the fractionation section to different than in the embodiment of FIG. 1. The overhead fraction comprises C6− hydrocarbons like the embodiment of FIG. 1. The reforming charge stream in line 124′ may comprise naphthenes and aromatics with six to ten carbon atoms and paraffins of seven to ten carbon atoms. The recycle stream in line 126′ may comprise hydrocarbons of eleven or more carbons atoms. In an exemplary embodiment, the reforming charge stream in line 124′ may comprise C6 to C10 naphthenes, C6 to C10 aromatics, and C7 to C10 paraffins, and the recycle stream in line 126′ may comprise C11+ paraffins. Hence, the cut in the overhead is taken between C6 paraffins and C6 naphthenes. The side cut is taken between C10 and C11 hydrocarbons. The recycle stream in line 126′ is recycled back to the hydrocracking reactor 110.

The reforming charge stream in line 124′ is charged to the reforming reactor in the reforming unit 130. In the reforming reactor 130, the reforming charge stream in line 124′ is contacted with the reforming catalyst to convert naphthenes and paraffins to aromatics in a reformed stream. The reformed stream comprising an increased concentration of aromatics leaves the reforming reactor in line 132′. In the embodiment as shown in FIG. 2, the C6 to C10 naphthenes, and C7 to C10 paraffins are reformed in the reforming reactor to produce C6 to C10 aromatics. The hydrocracked paraffinic stream in line 122 can be diverted through the reforming unit 130, with little conversion thereof.

The reformed stream in line 132′ may comprise hydrogen, light ends such as C5-hydrocarbons, BTX and C9+ aromatics. The reformed stream in line 132′ and the hydrocracked paraffinic stream in line 122 are charged to the NEP reactor 140. After paraffin conversion, a light paraffinic stream in line 142′ is discharged from the NEP reactor 140. The C9+ aromatics leave the NEP reactor 140 in the light paraffinic stream in line 142′. In an aspect, the light paraffinic stream in line 142′ as shown in FIG. 2 may comprise hydrogen, methane, benzene, toluene, xylenes, and C9+ aromatics along with the ethane and propane. The light paraffinic stream in line 142′ may be cooled and fed to the NEP separation section 150. A heavy stream comprising BTX and C9+ aromatics is discharged in line 158′ from the NEP separation section 150.

The heavy stream in line 158′ may be charged to the aromatic complex 190 to sperate aromatics, particularly benzene and xylenes. In the embodiment as shown in FIG. 2, the heavy stream in line 158′ may be fed to a splitter column in the aromatic complex to sperate light ends such as C5− hydrocarbons from the heavier hydrocarbons. The light ends stream may be taken in line 191 from the aromatic complex 190. The heavier hydrocarbons may be passed to the clay treater and processed in the aromatics complex 190 as earlier described in FIG. 1 to produce the benzene stream in line 192 and the selected paraxylene isomer stream in line 196. The rest of the process is same as previously described in FIG. 1.

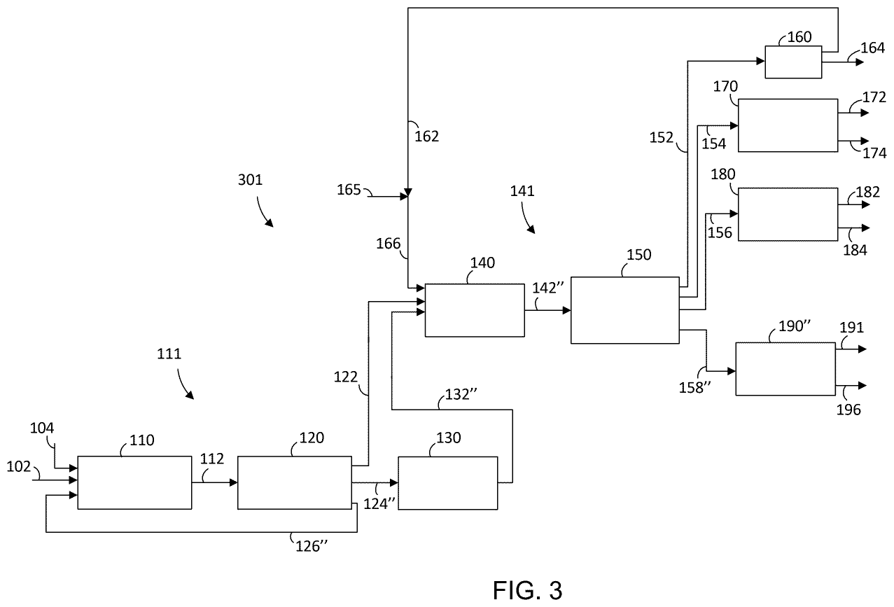

Another exemplary embodiment of the process for producing light olefins 301 is shown in FIG. 3. Elements in FIG. 3 with the same configuration as in FIG. 2 will have the same reference numeral as in FIG. 3. Elements in FIG. 3 which have a different configuration as the corresponding element in FIG. 3 will have the same reference numeral but designated with a double prime symbol (″). The configuration and operation of the embodiment of FIG. 3 is essentially the same as in FIG. 2 with the following exceptions.

In the embodiment shown in FIG. 3, the cut point of the reforming charge stream in line 124″ and the recycle stream in line 126″ are selected in the fractionation section. The hydrocracked paraffin stream in line 122 comprises C6− hydrocarbons as in the embodiment of FIGS. 1 and 2. The reforming charge stream in line 124″ may comprise naphthenes and aromatics with eight to ten carbon atoms and paraffins of nine to ten carbon atoms. The recycle stream in line 126″ may comprise two cuts. The first cut will be naphthenes and aromatics comprising six to 8 carbon atoms and paraffins comprising seven to eight carbon atoms. The second cut in the recycle stream will be hydrocarbons having at least 11 carbon atoms. Two columns may be used in the hydrocracking separation section 120 to produce the two recycle cuts. One column will separate the hydrocracked paraffin stream comprising the C6− hydrocarbons in line 122″ from a stream comprising naphthenes and aromatics of at least six carbon atoms. A second column can split a second overhead stream comprising naphthenes and aromatics comprising six to 8 carbon atoms and paraffins comprising seven to eight carbon atoms in a first recycle cut from a reforming charge stream in line 124″. The second cut comprising paraffins of at least 11 carbons can be produced in the bottom of the second column. The two recycle cuts can be combined to provide the recycle stream in line 126″ which is recycled back to the hydrocracking reactor 110. In an exemplary embodiment, the reforming charge stream in line 124″ may comprise C8 to C10 naphthenes and aromatics and C9 to C10 paraffins, and the recycle stream in line 126″ may comprise C6 to C8 naphthenes and aromatics, C7 to C8 paraffins and C11+ hydrocarbons.

In the embodiment as shown in FIG. 3, the benzene production is reduced as compared to the embodiment of FIG because benzene precursors are recycled to be hydrocracked instead of charged to the reformer. Particularly, C8 to C10 naphthenes, C8 to C10 aromatics, and C9 to C10 paraffins are selectively produced in in the reforming charge stream in line 124″ which is reformed in the reforming reactor to produce aromatics. The reformed stream predominantly comprises C8 to C10 aromatics for production of paraxylene in the aromatic complex 190. Benzene precursor, C6 to C8 naphthenes and aromatics are selectively taken in the recycle stream in line 126″ and recycled to hydrocracking unit 111 to be hydrocracked to smaller molecules, such as ethane and propane. Toluene precursors such as C7 naphthenes and C7 aromatics may be optionally processed either in the hydrocracking unit 111 or the reforming unit 130 to further adjust benzene production. If toluene is desired, C7 naphthenes and aromatics are admitted into the reforming charge stream. If toluene production is not desired, the C7 naphthenes and aromatics are left in the recycle stream in the first recycle cut. In this embodiment, the NEP unit 141 produces some BTX which is fed to the aromatic complex 190.

The reforming charge stream in line 124″ is charged to the reforming reactor in the reforming unit 130. In the reforming reactor, the reforming charge stream is contacted with the reforming catalyst to convert naphthenes to aromatics and produce a reformed stream. A reformed stream comprising an increased concentration of aromatics leaves the reforming reactor in line 132″. In the embodiment as shown in FIG. 3, the C8 to C10 naphthenes, and C9 to C10 paraffins are reformed in the reforming unit 130 to produce C8 to C10 aromatics.

The reformed stream in line 132″ may comprise hydrogen, light ends such as C5-hydrocarbons, C8-C10 aromatics including xylenes. The reformed stream in line 132″ and the hydrocracked paraffinic stream in line 122 are charged to the NEP reactor 140. After paraffin conversion, a light paraffinic stream in line 142″ is discharged from the NEP reactor 140. The C8-C10 aromatics leave the NEP reactor 140 in the light paraffinic stream in line 142″. In an aspect, the light paraffinic stream in line 142″ as shown in FIG. 3 may comprise hydrogen, methane, perhaps toluene, xylenes, and C8-C10 aromatics along with the ethane and propane. The light paraffinic stream in line 142″ may be cooled and fed to the NEP separation section 150. A heavy stream comprising perhaps toluene, xylenes, and C8-C10 aromatics is discharged in line 158″ from the NEP separation section 150.

The heavy stream in line 158″ may be charged to the aromatic complex 190 to sperate aromatics, particularly xylenes. In the embodiment as shown in FIG. 3, the heavy stream in line 158″ may be fed to the splitter column in the aromatic complex to sperate light ends such as C5− hydrocarbons from the heavier hydrocarbons. The light ends stream may be taken in line 191 from the aromatic complex 190. The heavier hydrocarbons may be passed to the clay treater and processed in the aromatics complex 190 as earlier described in FIG. 2 to produce the selected paraxylene isomer stream in line 196. The rest of the process is same as previously described in FIG. 2.

### Example

A yield estimation study was performed for the process as shown in FIG. 1. The details of the feed and products of the study are as below in Table 1. All units are in kilometric tons per annum.

TABLE 1

| Hydrocracking reactor 110 | | Reforming unit 130 | | NEP unit 141 | |
|---|---|---|---|---|---|
| Hydrocarbon | 5000 | Hydrocarbon feed | 5020 | Hydrocarbon feed | 4924 |
| feed | | from 110 | | from 130 | |
| Hydrogen feed | 144 | Product hydrogen | 96 | Hydrogen feed | 153 |
| Light ends product | 5020 | Product hydrocarbons | 4924 | Hydrogen product | 36 |
| (stream 124 to 130) | | (stream 132 to 141) | | | |
| | | (40% Aromatics and | | | |
| | | 40% C₇₋) | | | |
| | | | | C₁ product | 406 |
| | | | | C₂ product | 1750 |
| | | | | C₃ product | 695 |
| | | | | Aromatics product | 2188 |

Net results of the study from further conversion are as below in Table 2. All units are in kilometric tons per annum.

TABLE 2

| Feed | | Products | |
|---|---|---|---|
| Hydrocarbon feed (kmta) | 5000 | C₁ product (kmta) | 570 |
| Hydrogen feed (net over | 150 | Ethylene (kmta) | 1420 |
| all the units) (kmta) | | | |
| | | Propylene (kmta) | 692 |
| | | Benzene (kmta) | 427 |
| | | Paraxylene (kmta) | 1610 |
| | | Crude C₄ + Pygas + Pyoil (kmta) | 110 |

As evident from the results in Table 2, the process provided more than 80% yield of light olefins and aromatics from the hydrocarbon feed.

### Specific Embodiments

While the following is described in conjunction with specific embodiments, it will be understood that this description is intended to illustrate and not limit the scope of the preceding description and the appended claims.

A first embodiment of the present disclosure is a process for producing light olefins, comprising hydrocracking a hydrocarbon feed stream in a hydrocracking reactor over a hydrocracking catalyst in the presence of a hydrocracking hydrogen stream at hydrocracking conditions to produce a hydrocracked stream; reforming a reforming charge stream taken from the hydrocracked stream over a reforming catalyst in a reforming reactor to convert naphthenes to aromatics and produce a reformed stream; and charging the reformed stream to an NEP reactor to convert the reformed stream over an NEP catalyst in the presence of NEP hydrogen to produce a light paraffinic stream. An embodiment of the present disclosure is one, any or all of prior embodiments in this paragraph up through the first embodiment in this paragraph further comprising separating the light paraffinic stream to provide an ethane stream; and; cracking the ethane stream to produce an ethylene stream. An embodiment of the present disclosure is one, any or all of prior embodiments in this paragraph up through the first embodiment in this paragraph further comprising; separating a recycle stream from the hydrocracked stream; and recycling the recycle stream to the hydrocracking reactor. An embodiment of the present disclosure is one, any or all of prior embodiments in this paragraph up through the first embodiment in this paragraph wherein the reforming charge stream comprises naphthenes and aromatics with six carbon atoms. An embodiment of the present disclosure is one, any or all of prior embodiments in this paragraph up through the first embodiment in this paragraph wherein the recycle stream comprises hydrocarbons comprising at least 7 carbon atoms. An embodiment of the present disclosure is one, any or all of prior embodiments in this paragraph up through the first embodiment in this paragraph further comprising charging a hydrocracked paraffinic stream taken from the hydrocracked stream to the NEP reactor to convert the hydrocracked paraffinic stream over the NEP catalyst in the presence of NEP hydrogen to produce the light paraffinic stream. An embodiment of the present disclosure is one, any or all of prior embodiments in this paragraph up through the first embodiment in this paragraph wherein the hydrocracked paraffinic stream comprises paraffins with no more than 6 carbon atoms. An embodiment of the present disclosure is one, any or all of prior embodiments in this paragraph up through the first embodiment in this paragraph wherein the reforming charge stream comprises naphthenes and aromatics with six to ten carbon atoms. An embodiment of the present disclosure is one, any or all of prior embodiments in this paragraph up through the first embodiment in this paragraph wherein the recycle stream comprises paraffins comprising at least eleven carbon atoms. An embodiment of the present disclosure is one, any or all of prior embodiments in this paragraph up through the first embodiment in this paragraph wherein the reforming charge stream comprises naphthenes and aromatics with eight to ten carbon atoms. An embodiment of the present disclosure is one, any or all of prior embodiments in this paragraph up through the first embodiment in this paragraph wherein the recycle stream comprises naphthenes and aromatics comprising 6 carbon atoms. An embodiment of the present disclosure is one, any or all of prior embodiments in this paragraph up through the first embodiment in this paragraph further comprising separating an aromatic stream from the light paraffinic stream; and charging the aromatic stream to an aromatic complex to produce a xylene containing stream, the aromatics complex comprising at least one of an aromatics extraction unit, a BTX fractionation zone, a xylene fractionation zone, a transalkylation unit, and an isomerization unit.

A second embodiment of the present disclosure is a process for producing light olefins, comprising hydrocracking a hydrocarbon feed stream in a hydrocracking reactor over a hydrocracking catalyst in the presence of a hydrocracking hydrogen stream at hydrocracking conditions to produce a hydrocracked stream; separating the hydrocracked stream to provide a recycle stream and a reforming charge stream; reforming the reforming charge stream over a reforming catalyst in a reforming reactor to convert naphthenes to aromatics and produce a reformed stream; and charging the reformed stream to an NEP reactor to convert the reformed stream over an NEP catalyst in the presence of NEP hydrogen to produce a light paraffinic stream. An embodiment of the present disclosure is one, any or all of prior embodiments in this paragraph up through the second embodiment in this paragraph wherein the reforming charge stream comprises naphthenes and aromatics with six carbon atoms. An embodiment of the present disclosure is one, any or all of prior embodiments in this paragraph up through the second embodiment in this paragraph wherein the recycle stream comprises paraffins comprising at least 7 or 11 carbon atoms. An embodiment of the present disclosure is one, any or all of prior embodiments in this paragraph up through the second embodiment in this paragraph further comprising separating an aromatic stream from the light paraffinic stream; and charging the aromatic stream to an aromatic complex to produce a xylene containing stream, the aromatics complex comprising at least one of an aromatics extraction unit, a BTX fractionation zone, a xylene fractionation zone, a transalkylation unit, and an isomerization unit.

A third embodiment of the present disclosure is a process for producing light olefins, comprising hydrocracking a hydrocarbon feed stream in a hydrocracking reactor over a hydrocracking catalyst in the presence of a hydrocracking hydrogen stream at hydrocracking conditions to produce a hydrocracked stream; separating the hydrocracked stream to provide a reforming charge stream and a recycle stream; recycling the recycle stream to the hydrocracking reactor; reforming the reforming charge stream over a reforming catalyst in a reforming reactor to convert naphthenes to aromatics and produce a reformed stream, wherein the reforming charge stream comprises naphthenes and aromatics of at least 6 carbon atoms or at least 8 carbon atoms; and charging the reformed stream to an NEP reactor to convert the reformed stream over an NEP catalyst in the presence of NEP hydrogen to produce a light paraffinic stream. An embodiment of the present disclosure is one, any or all of prior embodiments in this paragraph up through the third embodiment in this paragraph further comprising charging a hydrocracked paraffinic stream taken from the hydrocracked stream to the NEP reactor to convert the hydrocracked paraffinic stream over the NEP catalyst in the presence of NEP hydrogen to produce the light paraffinic stream. An embodiment of the present disclosure is one, any or all of prior embodiments in this paragraph up through the third embodiment in this paragraph wherein the hydrocracked paraffinic stream comprises paraffins with no more than 6 carbon atoms. An embodiment of the present disclosure is one, any or all of prior embodiments in this paragraph up through the third embodiment in this paragraph further comprising separating an aromatic stream from the light paraffinic stream; and charging the aromatic stream to an aromatic complex to produce a xylene containing stream, the aromatics complex comprising at least one of an aromatics extraction unit, a BTX fractionation zone, a xylene fractionation zone, a transalkylation unit, and an isomerization unit.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for producing light olefins, comprising:
hydrocracking a hydrocarbon feed stream in a hydrocracking reactor over a hydrocracking catalyst in the presence of a hydrocracking hydrogen stream at hydrocracking conditions to produce a hydrocracked stream;
reforming a reforming charge stream taken from said hydrocracked stream over a reforming catalyst in a reforming unit to convert naphthenes to aromatics and produce a reformed stream; and
charging said reformed stream to an NEP reactor to convert said reformed stream over an NEP catalyst in the presence of NEP hydrogen to produce a light paraffinic stream.

2. The process of claim 1 further comprising:
separating said light paraffinic stream to provide an ethane stream; and;
cracking said ethane stream to produce an ethylene stream.

3. The process of claim 1 further comprising;
separating a recycle stream from said hydrocracked stream; and
recycling said recycle stream to the hydrocracking reactor.

4. The process of claim 3 wherein said reforming charge stream comprises naphthenes and aromatics with six carbon atoms.

5. The process of claim 4 wherein said recycle stream comprises hydrocarbons comprising at least 7 carbon atoms.

6. The process of claim 3 further comprising charging a hydrocracked paraffinic stream taken from said hydrocracked stream to said NEP reactor to convert said hydrocracked paraffinic stream over said NEP catalyst in the presence of NEP hydrogen to produce said light paraffinic stream.

7. The process of claim 6 wherein said hydrocracked paraffinic stream comprises paraffins with no more than 6 carbon atoms.

8. The process of claim 3 wherein said reforming charge stream comprises naphthenes and aromatics with six to ten carbon atoms.

9. The process of claim 8 wherein said recycle stream comprises paraffins comprising at least eleven carbon atoms.

10. The process of claim 3 wherein said reforming charge stream comprises naphthenes and aromatics with eight to ten carbon atoms.

11. The process of claim 10 wherein said recycle stream comprises naphthenes and aromatics comprising 6 carbon atoms.

12. The process of claim 1 further comprising:
separating an aromatic stream from said light paraffinic stream; and
charging said aromatic stream to an aromatic complex to produce a xylene containing stream, the aromatics complex comprising at least one of an aromatics extraction unit, a BTX fractionation zone, a xylene fractionation zone, a transalkylation unit, and an isomerization unit.

13. A process for producing light olefins, comprising:
hydrocracking a hydrocarbon feed stream in a hydrocracking reactor over a hydrocracking catalyst in the presence of a hydrocracking hydrogen stream at hydrocracking conditions to produce a hydrocracked stream;
separating said hydrocracked stream to provide a recycle stream and a reforming charge stream;
reforming said reforming charge stream over a reforming catalyst in a reforming unit to convert naphthenes to aromatics and produce a reformed stream; and
charging said reformed stream to an NEP reactor to convert said reformed stream over an NEP catalyst in the presence of NEP hydrogen to produce a light paraffinic stream.

14. The process of claim 13 wherein said reforming charge stream comprises naphthenes and aromatics with six carbon atoms.

15. The process of claim 15 wherein said recycle stream comprises paraffins comprising at least 7 or 11 carbon atoms.

16. The process of claim 13 further comprising:
separating an aromatic stream from said light paraffinic stream; and
charging said aromatic stream to an aromatic complex to produce a xylene containing stream, the aromatics complex comprising at least one of an aromatics extraction unit, a BTX fractionation zone, a xylene fractionation zone, a transalkylation unit, and an isomerization unit.

17. A process for producing light olefins, comprising:
hydrocracking a hydrocarbon feed stream in a hydrocracking reactor over a hydrocracking catalyst in the presence of a hydrocracking hydrogen stream at hydrocracking conditions to produce a hydrocracked stream;
separating said hydrocracked stream to provide a reforming charge stream and a recycle stream;
recycling said recycle stream to the hydrocracking reactor;
reforming said reforming charge stream over a reforming catalyst in a reforming unit to convert naphthenes to aromatics and produce a reformed stream, wherein said reforming charge stream comprises naphthenes and aromatics of at least 6 carbon atoms or at least 8 carbon atoms; and
charging said reformed stream to an NEP reactor to convert said reformed stream over an NEP catalyst in the presence of NEP hydrogen to produce a light paraffinic stream.

18. The process of claim 17 further comprising charging a hydrocracked paraffinic stream taken from said hydrocracked stream to said NEP reactor to convert said hydrocracked paraffinic stream over said NEP catalyst in the presence of NEP hydrogen to produce said light paraffinic stream.

19. The process of claim 18 wherein said hydrocracked paraffinic stream comprises paraffins with no more than 6 carbon atoms.

20. The process of claim 17 further comprising:
separating an aromatic stream from said light paraffinic stream; and
charging said aromatic stream to an aromatic complex to produce a xylene containing stream, the aromatics complex comprising at least one of an aromatics extraction unit, a BTX fractionation zone, a xylene fractionation zone, a transalkylation unit, and an isomerization unit.
